(19) United States
(12) Patent Application Publication
(10) Pub. No.: US 2026/0027796 A1
(43) Pub. Date: Jan. 29, 2026

(54) FILTER PRESSING SYSTEM AND FILTER PRESSING METHOD

(71) Applicant: CHEMLEX PTE. LTD., Singapore (SG)

(72) Inventors: Xiangxiang Li, Singapore (SG); Chao Lu, Singapore (SG)

(21) Appl. No.: 18/783,554

(22) Filed: Jul. 25, 2024

(51) Int. Cl. B30B 9/04; B30B 9/06; G01N 33/15

(52) U.S. Cl. CPC B30B 9/042; B01D 63/069; B01D 69/04; B01D 69/10; B30B 9/06; G01N 33/15

(57) ABSTRACT

The embodiment of the present disclosure discloses a filter pressing system and a filter pressing method, and the filter pressing system includes a filter pressing bottle group transfer module, a liquid transfer module, and a filter pressing transfer module, and the filter pressing bottle group transfer module is configured to drive a filter pressing bottle group carrier, and a first filter pressing bottle is connected to a second filter pressing bottle through a filter pressing membrane provided; the liquid transfer module is configured to drive a liquid transfer component filled with reaction liquid to move, and a controller controls the liquid transfer component to inject the reaction liquid into a target filter pressing bottle; the filter pressing transfer module is configured to drive a first clamping device to clamp the first filter pressing bottle and move it to a filter pressing position.

### FIELD

The present disclosure relates to the field of analysis and detection, and in particular to a filter pressing system and a filter pressing method.

### BACKGROUND OF THE DISCLOSURE

In the pharmaceutical synthesis experiment, it is necessary to filter press the insoluble substances in the reaction liquid and obtain a clear liquor for analysis and subsequent pharmaceutical synthesis treatment. In the prior art, a commonly used method is to manually absorb the reaction liquid from a test tube using a syringe, and then install a needle filter pressing head on the syringe head. The insoluble substances in the reaction liquid are filter pressed by pressing down on the syringe piston. Such an operation not only has low production efficiency and complex manual operation process, but also the hands of the experimenter are prone to contamination during the installation of the needle filter pressing head. Multiple sample transfers during the operation can also cause experimental errors; the entire process requires a high level of personnel quality, which does not meet the requirements of high-intensity operations and cannot undertake the automated synthesis process, making it unsuitable for automation.

### BRIEF SUMMARY OF THE DISCLOSURE

To address at least one disadvantages of the prior art above, the present disclosure provides a filter pressing system and a filter pressing method, and the filter pressing system achieves automated connection between multiple processes of filter pressing by provision of a multi-directional transfer module, achieving automated filter pressing; it solves the problem of low manual production efficiency, reduces filter pressing costs, improves filter pressing efficiency, and can avoid hand contamination by test personnel, meeting the requirements of high-intensity operations; multi-module collaboration work with dispersed flow direction shortens the spatial distance between operation steps in the filter pressing process, reduces the transportation and waiting time between operation steps, makes the flow between different operation steps more convenient, significantly improves the working efficiency of the filter pressing process, and can simultaneously meet the requirements of high-throughput experimental processing. The embodiment of the present disclosure is as follows:

On the one hand, a filter pressing system is provided, the filter pressing system includes:
a filter pressing bottle group transfer module, provided with a filter pressing position at one end thereof in a Y-axis direction, used to drive a filter pressing bottle group carrier to move in the Y-axis direction, the filter pressing bottle group carrier being used to load a filter pressing bottle group, the filter pressing bottle group including a first filter pressing bottle and a second filter pressing bottle, the first filter pressing bottle being connected to the second filter pressing bottle through a filter pressing membrane provided;
a liquid transfer module, used to drive a liquid transfer component filled with reaction liquid to move in a first predetermined direction, the first predetermined direction including an X-axis direction and a Y-axis direction, when the liquid transfer component moves above the filter pressing bottle group, the controller controlling the liquid transfer component to inject the reaction liquid into a target filter pressing bottle; the target filter pressing bottle being the first filter pressing bottle or the second filter pressing bottle; and
a filter pressing transfer module, used to drive a first clamping device to move in a second predetermined direction including an X-axis direction and a Z-axis direction to clamp the first filter pressing bottle and move to the filter pressing position, cooperate with the second filter pressing bottle that moves to the filter pressing position, and apply pressure to the second filter pressing bottle through the first filter pressing bottle to filter press the reaction liquid in the target filter pressing bottle through the filter pressing membrane.

In some embodiments, the filter pressing system further includes an analysis disk transfer module, used to drive an analysis disk to move in the Y-axis direction, and the analysis disk transfer module is adjacent to the filter pressing bottle group transfer module parallel to the Y-axis; and
the end of the analysis disk transfer module close to the filter pressing position is a filter pressing bottle group placement position.

In some embodiment, the filter pressing system further includes an analysis disk discharge component;
the analysis disk discharge component includes an analysis disk Y-axis transfer module, an analysis disk Z-axis transfer module and a second clamping device, and the second clamping device is provided on the analysis disk Z-axis transfer module;
the analysis disk transfer module is configured to drive the analysis disk to arrive at the filter pressing bottle group placement position;
the filter pressing transfer module drives the filter pressing bottle group to move to the filter pressing bottle group placement position to place the filter pressing bottle group on the analysis disk; and
the analysis disk Y-axis transfer module and the analysis disk Z-axis transfer module are linked to drive the second clamping device to move to the filter pressing bottle group placement position to clamp the analysis disk filled with the filter pressing bottle group to move to the next process.

In some embodiments, in case that a first carrying portion used to carry a first filter pressing membrane is provided at the bottom of the first filter pressing bottle, and the target filter pressing bottle is the second filter pressing bottle in case that the bottom of the second filter pressing bottle is sealed; and
in the case a carrier membrane is embedded at the bottom of the first filter pressing bottle, and a second carrying portion used to carry a second filter pressing membrane is provided away from the bottom of the second filter pressing bottle, the target filter pressing bottle is the first filter pressing bottle.

In some embodiments, there are at least two groups of the filter pressing bottle group transfer modules, and the two groups of filter pressing bottle group transfer modules are adjacent to the analysis disk transfer module parallel to the Y-axis.

In some embodiments, the liquid transfer module includes a liquid transfer X-axis transfer module and a liquid transfer Y-axis transfer module, and the liquid transfer X-axis transfer module is provided above the filter pressing bottle group transfer module; the liquid transfer Y-axis transfer module is adjacent to the analysis disk Y-axis transfer module parallel to the Y-axis, and the liquid transfer X-axis transfer module and the liquid transfer Y-axis transfer module are linked to drive the liquid transfer component to move above the filter pressing bottle group transfer module.

In some embodiments, the filter pressing transfer module includes a filter pressing X-axis transfer module and a filter pressing Z-axis transfer module, and the filter pressing X-axis transfer module is provided above the X-axis where the filter pressing position is; the first clamping device is provided on the filter pressing Z-axis transfer module, and the filter pressing X-axis transfer module and the filter pressing Z-axis transfer module are linked to drive the first clamping device to move.

In some embodiments, a liquid receiving component is provided directly below the liquid transfer component, and the liquid receiving component is configured to collect the reaction liquid dripped from the liquid transfer component.

In some embodiments, the liquid receiving component includes a liquid receiving tank, a driving mechanism, and an installation plate used to connect with the liquid transfer component, and the driving mechanism is configured to drive the liquid receiving tank to move.

In some embodiments, the filter pressing system further includes a support component, and the support component includes a support column and a support arm vertically provided with the support column;
the support column is configured to support the liquid transfer module, filter pressing transfer module, analysis disk discharge component and the support arm; and
the liquid transfer module and the analysis disk discharge component are provided on the support arm, and in sliding connection.

On the other hand, a filter pressing method is further provided, and the filter pressing method includes:
the liquid transfer module drives a liquid transfer component filled with reaction liquid to move above a filter pressing bottle group, and the controller controls the liquid transfer component to inject the reaction liquid into a target filter pressing bottle; the target filter pressing bottle is the first filter pressing bottle or the second filter pressing bottle;
the filter pressing bottle group transfer module drives the filter pressing bottle group carrier to move to the filter pressing position, and the filter pressing bottle group carrier is loaded with the second filter pressing bottle; and
the filter pressing transfer module drives a first clamping device to clamp the first filter pressing bottle and move to the filter pressing position, cooperate with the second filter pressing bottle that moves to the filter pressing position, and apply pressure to the second filter pressing bottle through the first filter pressing bottle to filter press the reaction liquid in the target filter pressing bottle through the filter pressing membrane.

By adopting the above embodiments of the present disclosure has the following advantages:

The present disclosure discloses a filter pressing system and a filter pressing method, and the filter pressing system includes a filter pressing bottle group transfer module, a liquid transfer module, and a filter pressing transfer module, and the filter pressing bottle group transfer module is configured to drive a filter pressing bottle group carrier, and the filter pressing bottle group carrier is configured to load the filter pressing bottle group; the filter pressing bottle group includes a first filter pressing bottle and a second filter pressing bottle, and the first filter pressing bottle is connected to the second filter pressing bottle through a filter pressing membrane provided; the liquid transfer module is configured to drive a liquid transfer component filled with reaction liquid to move, and a controller controls the liquid transfer component to inject the reaction liquid into a target filter pressing bottle; the target filter pressing bottle is the first filter pressing bottle or the second filter pressing bottle; the filter pressing transfer module is configured to drive a first clamping device to clamp the first filter pressing bottle and move it to a filter pressing position, cooperate with the second filter pressing bottle that moves to the filter pressing position, and apply pressure to the second filter pressing bottle through the first filter pressing bottle to filter press the reaction liquid in the target filter pressing bottle through the filter pressing membrane. The filter pressing system achieves automated connection between multiple processes of filter pressing by provision of a multi-directional transfer module, achieving automated filter pressing; it addresses the disadvantage of low manual production efficiency, reduces filter pressing costs, improves filter pressing efficiency, and can avoid contamination on hands of test personnel, meeting the requirements of high-intensity operations; multi-module collaboration work with dispersed flow direction shortens the spatial distance between operation steps in the filter pressing process, reduces the transportation and waiting time between operation steps, makes the flow between different operation steps more convenient, significantly improves the working efficiency of the filter pressing process, and can simultaneously meet the requirements of high-throughput experimental processing.

### BRIEF DESCRIPTION OF THE DRAWINGS

In order to describe the embodiments of the present disclosure more clearly, the drawings required for describing the embodiments will be introduced briefly. The drawings in the specification below are just some of the embodiments of the present disclosure.

FIG. 1 is a structural diagram of a filter pressing system provided in an embodiment of the present disclosure;

FIG. 2 is a structural diagram of a filter pressing bottle group transfer module and an analysis disk transfer module provided in an embodiment of the present disclosure;

FIG. 3 is a structural diagram of a filter pressing position and a filter pressing bottle group placement position provided in an embodiment of the present disclosure;

FIG. 4 is a structural diagram of a liquid transfer module provided in an embodiment of the present disclosure;

FIG. 5 is a structural diagram of a filter pressing transfer module provided in an embodiment of the present disclosure;

FIG. 6 is a structural diagram of an analysis disk discharge component provided in an embodiment of the present disclosure;

FIG. 7 is a structural diagram of a first filter pressing bottle, a second filter pressing bottle and a filter pressing bottle group in one group provided in an embodiment of the present disclosure;

FIG. 8 is a structural diagram of a liquid transfer component provided in an embodiment of the present disclosure;

FIG. 9 is a structural diagram of a liquid receiving component provided in an embodiment of the present disclosure;

FIG. 10 is a structural diagram of a first clamping device provided in an embodiment of the present disclosure; and

FIG. 11 is a structural diagram of a first clamping finger provided in an embodiment of the present disclosure.

Supplementary explanations are made below for the drawings:
11—filter pressing bottle group transfer module; 12—filter pressing position; 13—filter pressing bottle group carrier; 14—first filter pressing bottle; 15—second filter pressing bottle; 16—analysis disk transfer module; 17—analysis disk; 18—filter pressing bottle group placement position;
21—liquid transfer module; 211—liquid transfer X-axis transfer module; 212—liquid transfer Y-axis transfer module; 22—liquid transfer component;
31—filter pressing transfer module; 311—filter pressing X-axis transfer module; 312—filter pressing Z-axis transfer module; 32—first clamping device; 321—first grip; 322—first clamping finger; 3221—pressure bearing surface; 3222—holding surface;
4—analysis disk discharge component; 41—analysis disk Y-axis transfer module; 42—analysis disk Z-axis transfer module; 43—second clamping device;
5—liquid receiving component; 51—liquid receiving tank; 52—guide bar; 53—driving mechanism; 54—installation plate;
6—support component; 61—support column; 62—support arm;

### DETAILED DESCRIPTION OF THE DISCLOSURE

The embodiment of the present disclosure will be described completely and in detail below in conjunction with the drawings in an embodiment of the present disclosure. Apparently, the embodiments as described are simply parts of the embodiments of the present disclosure, rather than all of the embodiments.

The term “one embodiment” or “embodiment” referred here refers to a specific feature, structure, or characteristic that can be included in at least one implementation of the present disclosure. For specification of the present disclosure, it should be understood that orientation or position relations indicated by the terms “above”, “under”, “top”, “bottom”, etc. are based on the orientation or position relations shown in the drawings, and are only used to describe the present disclosure and simplify specification herein but not to indicate or imply that the device or component indicated must have a specific orientation and be constructed and operated in a specific orientation. Therefore, they shall not be construed as a limitation to the present disclosure. In addition, terms “first” and “second” are only used for specification, but cannot be understood to indicate or imply relative importance or implicitly indicate the number of embodiments described. Therefore, features defined as “first” and “second” may include one or more of these features explicitly or implicitly. Moreover, the terms, including “first”, “second” and the like, are intended to distinguish the similar objects, instead of describing a specific sequence or a precedence order. It should be understood that the data used in such a way can be exchanged where appropriate, and the embodiments of the present disclosure described herein can be implemented in addition to those illustrated or described here.

When a numerical range is disclosed herein, the above range is considered continuous and includes the minimum and maximum of the range, as well as each value between these minimum and maximum values. Furthermore, when the range refers to an integer, each integer between the minimum and the maximum of this range is included. In addition, when multiple ranges are provided to describe features or characteristics, these ranges can be merged. In other words, unless otherwise specified, all ranges disclosed herein shall be understood to include any and all sub-ranges included therein. For example, the specified range from “1 to 10” should be considered to include any and all sub-ranges between the minimum value 1 and the maximum 10. The exemplary sub-ranges of range from 1 to 10 include but are not limited to 1 to 6.1, 3.5 to 7.8, 5.5 to 10, etc.

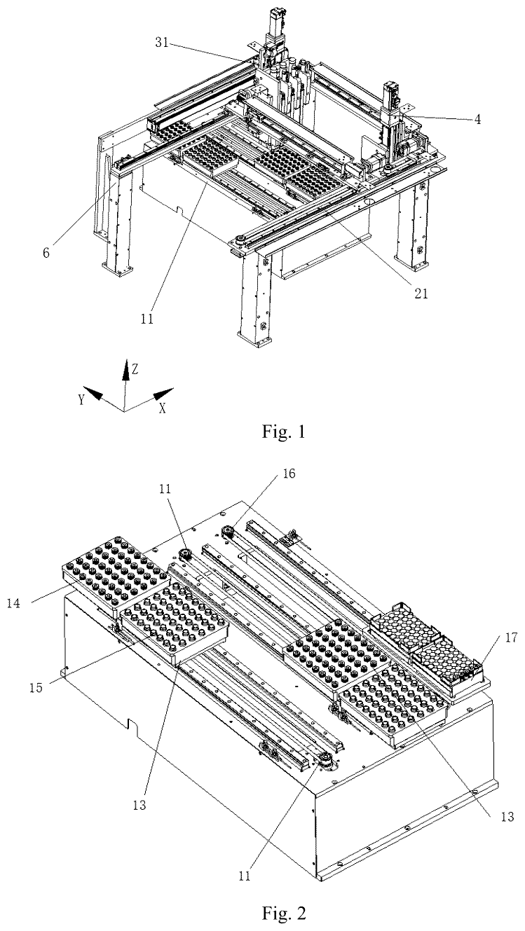

In one embodiment, referring to FIG. 1, the filter pressing system includes: a filter pressing bottle group transfer module 11, provided with a filter pressing position 12 at one end thereof in a Y-axis direction, used to drive a filter pressing bottle group carrier 13 to move in the Y-axis direction, the filter pressing bottle group carrier 13 being used to load a filter pressing bottle group, the filter pressing bottle group including a first filter pressing bottle 14 and a second filter pressing bottle 15, the first filter pressing bottle 14 being connected to the second filter pressing bottle 15 through a filter pressing membrane provided; in one embodiment, the first filter pressing bottle 14 is connected to the second filter pressing bottle 15 through a pressure filter membrane provided; in one embodiment, the filter pressing bottle group transfer module 11 can be a synchronous belt module, which can be driven in various ways and combinations, not limited to synchronous belt modules, but can also be driven by air cylinders, electric cylinders, or screw modules.

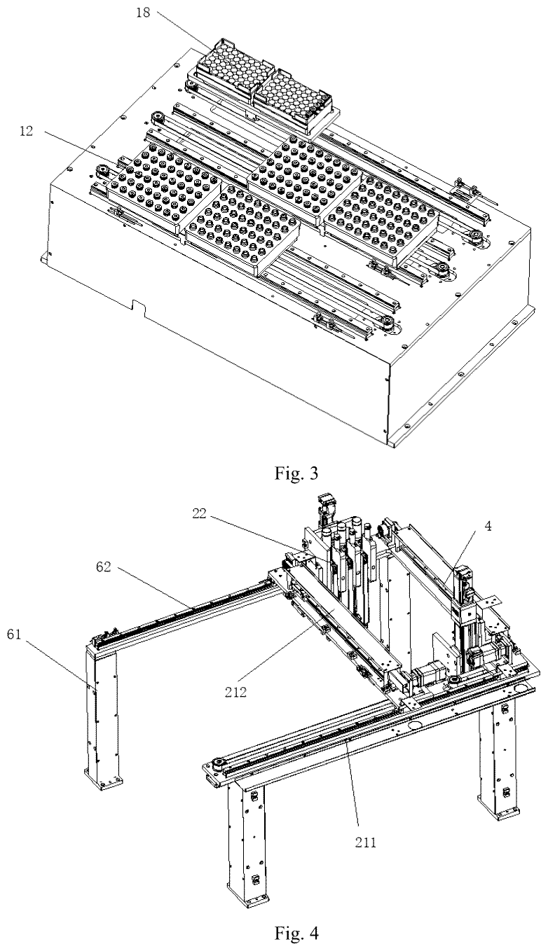

Referring to FIG. 3, the filter pressing position 12 is located near one end of the filter pressing bottle group transfer module 11. Firstly, a second pressure filtration bottle 15 is installed in the filter pressing bottle group carrier 13. Then, the filter pressing bottle group transfer module 11 drives the filter pressing bottle group carrier 13 to move to the filter pressing position 12. The filter pressing transfer module 31 clamps the first filter pressing bottle 14 and completes the filter pressing at the filter pressing position 12.

In one embodiment, referring to FIG. 7, the first filter pressing bottle 14 can be an inner filter pressing bottle, the second filter pressing bottle 15 can be an outer filter pressing bottle, and the inner filter pressing bottle is nested inside the outer filter pressing bottle. In one embodiment, a first carrying portion used to carry a filter pressing membrane is provided at the bottom of the first filter pressing bottle 14, and a reinforcing rib is also provided at the connection between the filter pressing membrane and the first filter pressing bottle. The filter pressing membrane will not directly contact the first carrying portion. Placing the filter pressing membrane on the first carrying portion can improve the accuracy of the position where the filter pressing membrane is fixed, and the accuracy of filter pressing is ensured. The bottom of the second filter pressing bottle 15 is sealed. In such an implementation, the second filter pressing bottle 15 is filled with reaction liquid, the first filter pressing bottle 14 is pressed into the second filter pressing bottle 15, and the reaction liquid enters the first filter pressing bottle 14 through the filter pressing membrane. In another embodiment, the bottom of the first filter pressing bottle 14 is sealed by the carrier membrane, and a second carrying portion used to carry the filter pressing membrane is provided away from the bottom of the second filter pressing bottle 15. In such an implementation, the second filter pressing bottle 15 is filled with reaction liquid, the first filter pressing bottle 14 is pressed into the second filter pressing bottle 15, and the reaction liquid enters the second filter pressing bottle 14 through the filter pressing membrane. The above filter pressing membrane is a unidirectional filter membrane, which can cause the reaction liquid filled in the first filter pressing bottle 14 to flow towards the second filter pressing bottle 15, or the reaction liquid filled in the second filter pressing bottle 15 to flow towards the first filter pressing bottle 14. The above two implementations can use unidirectional filter membranes in different directions; the material of the unidirectional filter membrane can be different according to the characteristics of different samples, such as polytetrafluoroethylene or polyamide.

In one embodiment, referring to FIG. 2, the filter pressing bottle group carrier 13 includes a first filter pressing bottle carrier and a second filter pressing bottle carrier, and there are at least two groups of filter pressing bottle group transfer modules 11. The first filter pressing bottle carrier and the second filter pressing bottle carrier can be provided on the same filter pressing bottle group transfer module 11, or they can be respectively provided on different filter pressing bottle group transfer modules 11. The first filter pressing bottle carrier and the second filter pressing bottle carrier are divided into multiple accommodating chambers, and the outer contour of the accommodating chamber is slightly larger than the outer diameter of the first filter pressing bottle 14 and the second filter pressing bottle 15. Preferably, the shape of the accommodating chamber is circular or square, which facilitates the placement of the first filter pressing bottle 14 and the second filter pressing bottle 15 into the accommodating chamber, making the placement of the first filter pressing bottle 14 and the second filter pressing bottle 15 more stable and less prone to overturning.

The liquid transfer module 21 is configured to drive the liquid transfer component 22 filled with reaction liquid to move in the first predetermined direction, and the first predetermined direction includes an X-axis direction and a Y-axis direction. When the liquid transfer component 22 moves above the filter pressing bottle group, the controller controls the liquid transfer component 22 to inject the reaction liquid into the target filter pressing bottle; the target filter pressing bottle is the first filter pressing bottle or the second filter pressing bottle; in one embodiment, when the bottom of the first filter pressing bottle is provided with a filter pressing membrane, the liquid transfer component 22 injects the reaction liquid into the second filter pressing bottle 15; when the bottom of the first filter pressing bottle 14 is provided with a carrier membrane, but the second filter pressing bottle 15 is provided with a filter pressing membrane away from the bottom, the liquid transfer component 22 injects the reaction liquid into the first filter pressing bottle 14; and the filter pressing bottle group transfer module 11 includes a first motor and a first synchronous belt, and the filter pressing bottle group carrier 13 carries the second filter pressing bottle 15, and the filter pressing bottle group carrier 13 on the first synchronous belt moves to the filter pressing position 12, to move the second filter pressing bottle 15 carried in the filter pressing bottle group carrier 13 to the filter pressing position 12;

The liquid transfer component 22 is controlled by the controller to inject the reaction liquid into the target filter pressing bottle. The controller serves as the control center of the entire filter pressing system equipment, and is configured to issue work instructions to the corresponding modules in good time to achieve collaborative work between different modules. For example, the controller can be one or more of PLC, microcomputer, computer control software, etc. The liquid transfer component 22 includes a pipette and a Tip head connected to the pipette. The Tip head, also known as a pipette head or liquid transfer head, is an experimental liquid transfer suction head and belongs to a consumable material that can store a certain capacity of liquid. The Tip head is placed in the Tip head compartment, and the tip of the pipette is provided with a joint part that cooperates with the Tip head. The pipette moves to the Tip head compartment and provides a downward force through the joint part to connect with the Tip head. After the Tip head is installed on the pipette, the pipette provides a suction force to suck the reaction liquid into the Tip head for storage. Then, the liquid transfer module 21 drives the liquid transfer component 22 filled with the reaction liquid to move to the target filter pressing bottle. The pipette spits out the liquid, and the Tip head injects the stored reaction liquid into the target filter pressing bottle to complete the suction and spitting operation; there may be multiple groups of liquid transfer components, for example, three groups, which can correspond to three different solutions. It can achieve simultaneous liquid transfer operations for different solutions, meet high flux requirements, and achieve batch liquid transfer operations. The pipette can be replaced with an injection pump or peristaltic pump.

In other embodiments, the first predetermined direction mentioned above may include the X-axis direction, Y-axis direction, and Z-axis direction. As an example, the liquid transfer module 21 drives the liquid transfer component 22 filled with the reaction liquid to move along the X-axis direction and Y-axis direction to the target filter pressing bottle. The liquid transfer module 21 can drive the Tip head to move along the Z-axis direction near the bottleneck of the target filter pressing bottle through the controller of the liquid transfer component 22, to complete the suction and spitting operation. The X-axis, Y-axis, and Z-axis directions mentioned in the present disclosure are perpendicular to each other.

The filter pressing transfer module 31 is configured to drive a first clamping device 32 to move in a second predetermined direction including an X-axis direction and a Z-axis direction to clamp the first filter pressing bottle 14 and move to the filter pressing position 12, cooperate with the second filter pressing bottle 15 that moves to the filter pressing position 12, and apply pressure to the second filter pressing bottle 15 through the first filter pressing bottle 14 to filter press the reaction liquid in the target filter pressing bottle through the filter pressing membrane. In one embodiment, when the bottom of the first filter pressing bottle 14 is provided with a filter pressing membrane, the first filter pressing bottle 14 applies pressure to the second filter pressing bottle 15 and the reaction liquid in the second filter pressing bottle 15 is filter pressed by the filter pressing membrane to enter the first filter pressing bottle 14; when the bottom of the first filter pressing bottle 14 is provided with a carrier membrane, but the second filter pressing bottle 15 is provided with a filter pressing membrane away from the bottom, the first filter pressing bottle 14 applies pressure to the second filter pressing bottle 15 and the reaction liquid in the first filter pressing bottle 14 is filter pressed by the filter pressing membrane to enter the second filter pressing bottle 15.

In one embodiment, the working mode of the filter pressing system is as follows: the liquid transfer module 21 drives the liquid transfer component 22 filled with the reaction liquid to move above the filter pressing bottle group, and the controller controls the liquid transfer component 22 to inject the reaction liquid into the second filter pressing bottle 15; the filter pressing bottle group carrier 13 is loaded with the second filter pressing bottle 15 injected with the reaction liquid. The filter pressing bottle group transfer module 11 drives the filter pressing bottle group carrier 13 to move to the filter pressing position 12; the filter pressing transfer module 31 drives the first clamping device 32 to clamp the first filter pressing bottle 14 and move it to the filter pressing position 12, cooperate with the second filter pressing bottle 15 located at the filter pressing position 12 and apply pressure to the second filter pressing bottle 15 through the first filter pressing bottle 14, and the reaction liquid in the second filter pressing bottle 15 enters the first filter pressing bottle 14 through the filter pressing membrane, completing the filter pressing. In another embodiment, the working mode of the filter pressing system is as follows: the liquid transfer module 21 drives the liquid transfer component 22 filled with the reaction liquid to move above the filter pressing bottle group, and the controller controls the liquid transfer component 22 to inject the reaction liquid into the first filter pressing bottle 14; the filter pressing bottle group carrier 13 is loaded with the second filter pressing bottle 15. The filter pressing bottle group transfer module 11 drives the filter pressing bottle group carrier 13 to move to the filter pressing position 12; the filter pressing transfer module 31 drives the first clamping device 32 to clamp the first filter pressing bottle 14 and move it to the filter pressing position 12, cooperate with the second filter pressing bottle 15 located at the filter pressing position 12 and apply pressure to the second filter pressing bottle 15 through the first filter pressing bottle 14, and the reaction liquid in the first filter pressing bottle 14 enters the second filter pressing bottle 15 through the filter pressing membrane, completing the filter pressing. The present disclosure is not limited to the order of the above operations, and can also choose an appropriate judgment order or perform several operations and steps simultaneously as needed, and this is not limited by the present disclosure. The filter pressing system achieves automated connection between multiple processes of filter pressing by provision of a multi-directional transfer module, achieving automated filter pressing; it solves the problem of low manual production efficiency, reduces filter pressing costs, improves filter pressing efficiency, and can avoid hand contamination by test personnel, meeting the requirements of high-intensity operations; multi-module collaboration work with dispersed flow direction shortens the spatial distance between operation steps in the filter pressing process, reduces the transportation and waiting time between operation steps, makes the flow between different operation steps more convenient, significantly improves the working efficiency of the filter pressing process, and can simultaneously meet the requirements of high-throughput experimental processing.

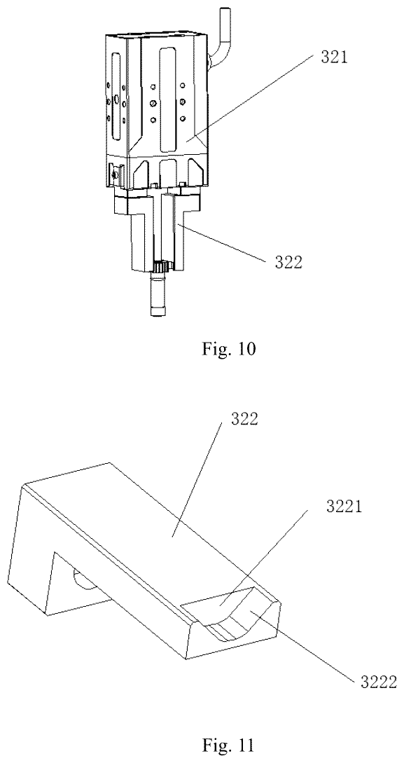

In one embodiment, referring to FIG. 10 and FIG. 11, the first clamping device 32 includes a first grip 321 and a first clamping finger 322; the first grip 321 can be an electrical claw or a gas claw, and the first clamping finger 322 is installed on the first grip 321; the first clamping finger 322 is controlled to open/close through the first grip 321; the first clamping finger is L-shaped, and there are at least two first clamping fingers; the first clamping fingers 322 corresponding to each other can be arranged to be able to approach each other to clamp the first filter pressing bottle 14 or the second filter pressing bottle 15, and to be able to move away from each other to release the first filter pressing bottle 14 or the second filter pressing bottle 15. In one embodiment, the first clamping finger 322 includes an installation plate and a clamping plate connected to the installation plate; the shorter side of the first clamping finger 322 that protrudes outwards is the installation plate, and a filter pressing part is provided at the end of the clamping plate away from the installation plate; the filter pressing part can define, for example, a portion of a spatial structure that can be used to clamp the first filter pressing bottle 14 or the second filter pressing bottle 15, and to press the first filter pressing bottle 14 down into the second filter pressing bottle 15, such as a groove, chamber, blind hole, and other structures. In some embodiments, the filter pressing part can be a groove, which includes a pressure bearing surface 3221 and a holding surface 3222. The holding surface 3222 is configured to clamp the first filter pressing bottle 14, and the two grooves cooperate with each other to form a cavity structure to clamp the first filter pressing bottle 14. Preferably, the shape of the cavity structure is polygonal, ensuring coaxiality, and the pressure bearing surface 3221 is configured to press down the first filter pressing bottle 14 down into the second filter pressing bottle 15 to withstand axial pressure. Clamping is made more stable, less prone to incline.

In one embodiment, referring to FIGS. 1 and 2, the filter pressing system further includes an analysis disk transfer module 16, used to drive an analysis disk 17 to move in the Y-axis direction, and the analysis disk transfer module 16 is adjacent to the filter pressing bottle group transfer module 11 parallel to the Y-axis;

The end of the analysis disk transfer module 16 close to the filter pressing position 12 is a filter pressing bottle group placement position 18.

The analysis disk transfer module 16 can be a synchronous belt module, which can be driven by multiple ways and combination thereof, not limited to the synchronous belt module, but can be driven by air cylinder, electrical cylinder or lead screw module. Preferably, the analysis disk transfer module 16 includes a second motor and a second synchronous belt. The analysis disk 17 is placed on the second synchronous belt and the second synchronous belt is driven by the second motor to move along the Y-axis direction, to drive the analysis disk 17 located on the second synchronous belt to move. The analysis disk 17 is divided into several accommodating chambers for loading the filter pressing bottle group. The analysis disk transfer module 16 drives the analysis disk 17 loaded with the filter pressing bottle group to move; the outer contour of the accommodating chamber is slightly larger than the outer diameter of the second filter pressing bottle 15. Preferably, the accommodating chamber is circular or square, which facilitates the placement of the filter pressing bottle group into the accommodating chamber, making the placement of the filter pressing bottle group more stable and less prone to overturning.

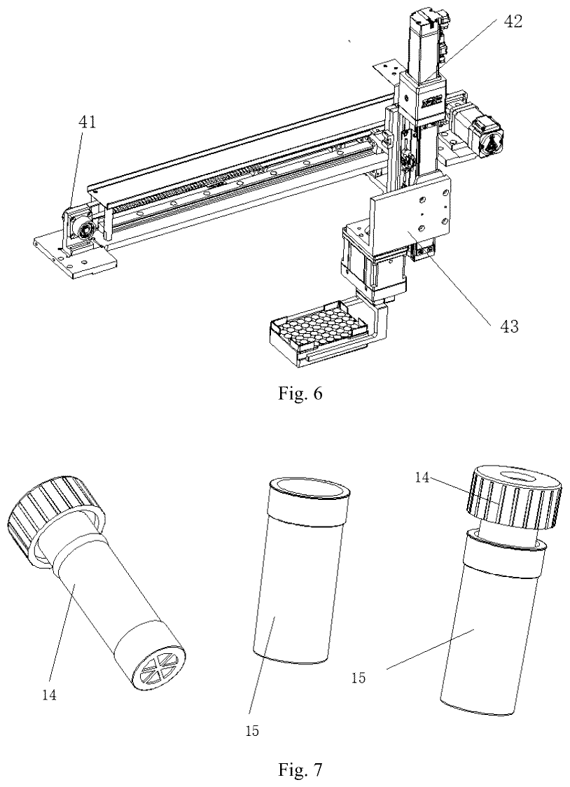

In one embodiment, referring to FIG. 6, the filter pressing system further includes an analysis disk discharge component 4;

The analysis disk discharge component 4 includes an analysis disk Y-axis transfer module 41, an analysis disk Z-axis transfer module 42 and a second clamping device 43, and the second clamping device 43 is provided on the analysis disk Z-axis transfer module 42; in one embodiment, the analysis disk Z-axis transfer module 42 and the second clamping device 43 are arranged in parallel along the Z-axis direction;

The analysis disk transfer module 16 drives the analysis disk 17 to arrive at the filter pressing bottle group placement position 18; in one embodiment, the filter pressing bottle group placement position 18 and the filter pressing position 12 are arranged co-axially along the X-axis, which facilitates the filter pressing transfer module to transfer the filter pressing bottle group to the analysis disk;

The filter pressing transfer module 31 drives the filter pressing bottle group to move to the filter pressing bottle group placement position 18 to place the filter pressing bottle group on the analysis disk 17;

The analysis disk Y-axis transfer module 41 and the analysis disk Z-axis transfer module 42 are linked to drive the second clamping device 43 to move to the filter pressing bottle group placement position 18 to clamp the analysis disk 17 filled with the filter pressing bottle group to move to the next process. In one embodiment, the analysis disk discharge component 4 moves in such a specific manner that, the analysis disk Y-axis transfer module 41 drives the analysis disk Z-axis transfer module 42 to move in the Y-axis direction, to drive the second clamping device 43 provided on the analysis disk Z-axis transfer module 42 to move along the Y-axis direction. While the analysis disk Z-axis transfer module 42 moves in the Y-axis direction, the analysis disk Z-axis transfer module 42 can also drive the second clamping device 43 synchronously to move along the Z-axis direction.

In one embodiment, referring to FIG. 6, the second clamping device 43 includes a second grip and a second clamping finger. The second grip can be an electric claw or a gas claw, and the opening and closing motion of the second clamping finger is controlled by the second grip. The second clamping finger is L-shaped, and there are two second clamping fingers. The two second clamping fingers work together to clamp the analysis disk 17. In one embodiment, the second clamping finger includes a connecting arm and a clamping arm, and the connecting arm is in fit connection with the second grip. The clamping arm is configured to clamp the analysis disk 17, and the outer wall of the analysis disk 17 is sunken inwards to form a clamping groove. The clamping groove and the clamping arm work together, and the clamping arm clamps the analysis disk 17 more stably, less prone to fall off or incline.

In one embodiment, the entire specific movement method for completing the analysis and discharge operation is as follows: the analysis disk transfer module 16 drives the analysis disk 17 to arrive at the filter pressing bottle group placement position 18, and then the first clamp device 32 is driven to move through the filter pressing transfer module 31 to clamp the filter pressing bottle group that has completed the filter pressing at the filter pressing position 12, and, the filter pressing transfer module 31 includes a filter pressing X-axis transfer module 311 and a filter pressing Z-axis transfer module 312. The filter pressing X-axis transfer module 311 drives the filter pressing Z-axis transfer module 312 to move to the filter pressing position 12 in the X-axis direction, and the filter pressing Z-axis transfer module 312 drives the first clamping device 32 to move along the Z-axis direction and clamp the filter pressing bottle group with filter pressing completed on the filter pressing position 12. The filter pressing X-axis transfer module 311 moves to the filter pressing bottle group placement position 18 along the X-axis direction, and places the filter pressing bottle group on the analysis disk 17, then the analysis disk Z-axis transfer module 42 is driven through the analysis disk Y-axis transfer module 41 to move to the filter pressing bottle group placement position 18 along the Y-axis direction, and the analysis disk Z-axis transfer module 42 drives the second clamping device 43 to move along the Z-axis direction to clamp the analysis disk 17 filled with filter pressing bottles, finally the analysis disk 17 clamped with the filter pressing bottle group is driven through the analysis disk Y-axis transfer module 41 to move to the next process, completing analysis and discharge. The present disclosure is not limited to the order of the above operations, and can also choose an appropriate judgment order or perform several operations and steps simultaneously as needed, and this is not limited by the present disclosure. Through provision of the analysis disk discharge component 4 beside the filter pressing device, the filter-pressed filter pressing bottle group filled with filter pressing liquid after filter pressing is completed is moved to the next process, which can save time, improve efficiency and avoid the movement of the filter pressing bottle group by experimenters. It realizes automated analysis operation, solves the problem of low manual production efficiency, reduces analysis costs, improves analysis efficiency, and can avoid hand contamination by test personnel, meeting the requirements of high-intensity operations.

In one embodiment, referring to FIG. 7, in case that a first carrying portion used to carry a first filter pressing membrane is provided at the bottom of the first filter pressing bottle 14, and the bottom of the second filter pressing bottle 15 is sealed, the target filter pressing bottle is the second filter pressing bottle 15; and
in the case a carrier membrane is embedded at the bottom of the first filter pressing bottle 14, and a second carrying portion used to carry a second filter pressing membrane is provided away from the bottom of the second filter pressing bottle 15, the target filter pressing bottle is the first filter pressing bottle 14.

The first filter pressing membrane and the second filter pressing membrane are both unidirectional filter pressing membranes, and their directions are different; the first carrying portion and the second carrying portion can be formed by crossing multiple support rods, or they can be support components formed in other ways that allow the reaction liquid to pass through, without any limitation here.

When the filter pressing bottle group is arranged in such a way, a first carrying portion used to carry a first filter pressing membrane is provided at the bottom of the first filter pressing bottle 14, the bottom of the second filter pressing bottle 15 is sealed. At this point, the liquid transfer module 21 drives the liquid transfer component 22 filled with the reaction liquid to move above the filter pressing bottle group, and the controller controls the liquid transfer component 22 to inject the reaction liquid into the second filter pressing bottle 15; the filter pressing bottle group carrier 13 is loaded with the second filter pressing bottle 15 injected with the reaction liquid. The filter pressing bottle group transfer module 11 drives the filter pressing bottle group carrier 13 to move to the filter pressing position 12; the filter pressing transfer module 31 drives the first clamping device 32 to clamp the first filter pressing bottle 14 and move it to the filter pressing position 12, cooperate with the second filter pressing bottle 15 located at the filter pressing position 12 and apply pressure to the second filter pressing bottle 15 through the first filter pressing bottle 14, and the reaction liquid in the second filter pressing bottle 15 is filter pressed into the first filter pressing bottle 14 through the filter pressing membrane, completing the filter pressing.

When the filter pressing bottle group is arranged in such a way, a carrier membrane is embedded at the bottom of the first filter pressing bottle 14, and a second carrying portion used to carry a second filter pressing membrane is provided away from the bottom of the second filter pressing bottle 15. At this point, the liquid transfer module 21 drives the liquid transfer component 22 filled with the reaction liquid to move above the filter pressing bottle group, and the controller controls the liquid transfer component 22 to inject the reaction liquid into the first filter pressing bottle 14; the filter pressing bottle group transfer module 11 drives the filter pressing bottle group carrier 13 loaded with the second filter pressing bottle 15 without reaction liquid injected to the filter pressing position 12; the filter pressing transfer module 31 drives the first clamping device 32 to clamp the first filter pressing bottle 14 and move it to the filter pressing position 12, cooperate with the second filter pressing bottle 15 located at the filter pressing position 12 and apply pressure to the second filter pressing bottle 15 through the first filter pressing bottle 14, and the reaction liquid in the first filter pressing bottle 14 is filter pressed into the second filter pressing bottle 15 through the filter pressing membrane, completing the filter pressing.

In one embodiment, referring to FIG. 3, there are at least two groups of the filter pressing bottle group transfer modules 11, and the two groups of filter pressing bottle group transfer modules 11 are adjacent to the analysis disk transfer module 16 parallel to the Y-axis. In one embodiment, a filter pressing position 12 is provided at one end of at least one group of filter pressing bottle group transfer modules 11, and, a first filter pressing bottle carrier can be placed on one group of filter pressing bottle group transfer modules 11, and a second filter pressing bottle carrier can be placed on the other group of filter pressing bottle group transfer modules 11. Alternatively, both the first filter pressing bottle carrier and second filter pressing bottle carrier can be placed on either group of filter pressing bottle group transfer modules 11 simultaneously; by provision of two groups of filter pressing bottle group transfer modules 11, when the first filter pressing bottle 14 and/or the second filter pressing bottle 15 loaded on one group of filter pressing bottle group transfer modules 11 are used up, the next group can be used directly to save time and improve efficiency.

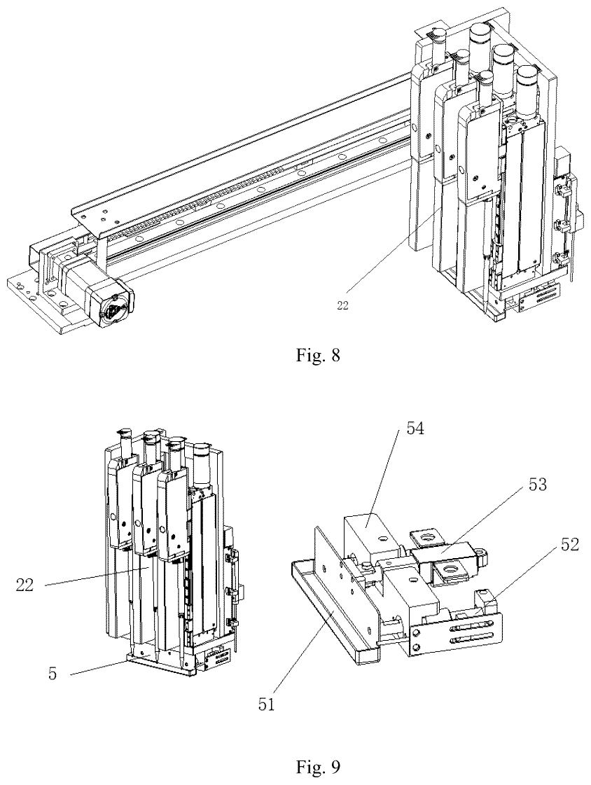

In one embodiment, referring to FIG. 4 and FIG. 8, the liquid transfer module 21 includes a liquid transfer X-axis transfer module 211 and a liquid transfer Y-axis transfer module 212, and the liquid transfer X-axis transfer module 211 is provided above the filter pressing bottle group transfer module 11; in one embodiment, the liquid transfer X-axis transfer module 211 can also be provided on the side close to the filter pressing transfer module 31, provided on the support arm 62. The liquid transfer Y-axis transfer module 212 and the analysis disk Y-axis transfer module 41 are adjacent, parallel to the Y-axis, and the liquid transfer Y-axis transfer module 212 is driven to move along the X-axis direction through the liquid transfer X-axis transfer module 211; while the liquid transfer Y-axis transfer module 212 moves along the X-axis direction, the liquid transfer Y-axis transfer module 212 synchronously drives the liquid transfer component 22 to move in the Y-axis direction, and the liquid transfer X-axis transfer module 211 and the liquid transfer Y-axis transfer module 212 are linked to drive the liquid transfer component 22 to move above at least two groups of filter pressing bottle group transfer modules 11. The movement is made more accurately and stably.

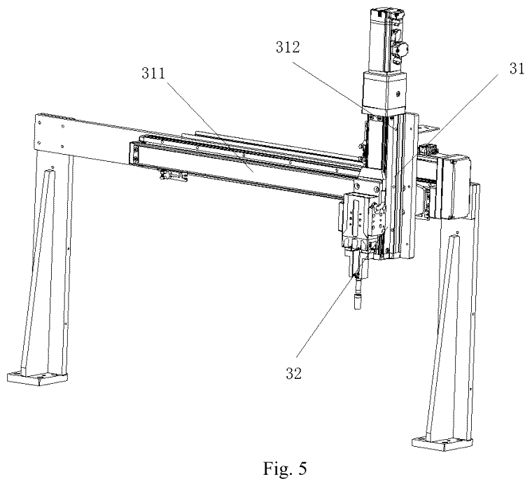

In one embodiment, referring to FIG. 5, the filter pressing transfer module 31 includes a filter pressing X-axis transfer module 311 and a filter pressing Z-axis transfer module 312. The filter pressing X-axis transfer module 311 is provided above the X-axis where the filter pressing position 12 is located, the filter pressing X-axis transfer module 311 and the liquid transfer X-axis transfer module 211 are provided parallel to the X-axis at both ends of the workbench, and a first clamping device 32 is provided on the filter pressing Z-axis transfer module 312. In one embodiment, the filter pressing Z-axis transfer module 312 and the first clamping device 32 are provided in parallel along the Z-axis direction; the filter pressing X-axis transfer module 311 drives the filter pressing Z-axis transfer module 312 to move along the X-axis direction. When the filter pressing Z-axis transfer module 312 moves along the X-axis direction, the filter pressing Z-axis transfer module 312 also synchronously drives the first clamping device 32 to move along the Z-axis direction, and the filter pressing X-axis transfer module 311 and the filter pressing Z-axis transfer module 312 are linked to drive the first clamping device 32 to move. The movement is made more accurately and stably.

In one embodiment, referring to FIG. 9, a liquid receiving component 5 is provided directly below the liquid transfer component 22. The liquid receiving component 5 is configured to collect the reaction liquid dripped by the liquid transfer component 22, and the liquid receiving component 5 moves synchronously with the liquid transfer component to facilitate the liquid transfer operation of the liquid transfer component throughout the entire working area. The liquid receiving component 5 collects the dripped liquid in time to avoid secondary contamination of the reaction liquid.

The liquid receiving component 5 includes a liquid receiving tank 51, a driving mechanism 53, and an installation plate 54 used to connect with the liquid transfer component 22, and the driving mechanism 53 is configured to drive the liquid receiving tank 51 to move.

In one embodiment, the liquid receiving component 5 further includes a guide bar 52; the guide bar 52 and the driving mechanism 53 are provided in parallel at both ends of the liquid receiving tank 51, and the driving mechanism 53 drives the guide bar 52 to act on the liquid receiving tank 51. The guide bar 52 acts on the liquid receiving tank 51 to make horizontal movement, and the receiving tank 51 moves more stably.

In one embodiment, referring to FIG. 1, the filter pressing system further includes a support component 6, and the support component 6 includes a support column 61 and a support arm 62 vertically arranged with the support column 61.

The support column 61 is configured to support the liquid transfer module 21, filter pressing transfer module 31, analysis disk discharge component 4 and the support arm 62.

The liquid transfer module 21 and the analysis disk discharge component 4 are provided in the support arm 62, and in sliding connection; in one embodiment, both ends of the liquid transfer Y-axis transfer module 212 and the analysis disk Y-axis transfer module 41 are in sliding connection on two support arms 62. The sliding connection consists of one having a raised slide rail and the other having a chute that matches the slide rail; it is also possible to provide a chute for one of the two, and a sliding block for the other; in addition, the analysis disk discharge component 4 is provided on the support arm 62 and the liquid transfer X-axis transfer module 211, which saves space and has a simple assembly relationship.

In one embodiment, the filter pressing system also includes a dilution component, which is configured to hold diluent. An installation plate is installed on the liquid transfer module 21, in which one side of the installation plate is installed with a liquid transfer component 22 and the other side is installed with a dilution component. If dilution is required during the filter pressing step, the dilution component will inject a corresponding amount of diluent under the control of the upper computer. If dilution is not required during the filter pressing step, the dilution component will not be started, and both the liquid transfer component and the dilution component will be configured to improve production efficiency, reduce equipment footprint, and achieve batch processing of liquid transfer or dilution. The dilution component is moved in such a specific manner: the liquid transfer Y-axis transfer module 212 is driven to move along the X-axis direction through the liquid transfer X-axis transfer module 211; while the liquid transfer Y-axis transfer module 212 moves along the X-axis direction, the liquid transfer Y-axis transfer module 212 synchronously drives the dilution component to move in the Y-axis direction, and the liquid transfer X-axis transfer module 211 and the liquid transfer Y-axis transfer module 212 are linked to drive the dilution component to move above the filter pressing bottle group transfer module 11. Therefore, the liquid transfer X-axis transfer module 211 and the liquid transfer Y-axis transfer module 212 are linked to drive the dilution component to inject the diluent into the target filter pressing bottle for dilution operation. There may be multiple groups of liquid transfer components, for example, three groups, which can complete dilution operation at the same time. A liquid receiving component 5 can also be installed directly below the dilution component. The liquid receiving component 5 is configured to collect the reaction liquid dripped from the dilution component, which can effectively avoid secondary contamination of the diluent.

The dilution component is controlled by a controller to inject diluent into the target filter pressing bottle, completing the dilution operation. It realizes automated dilution operation, solves the problem of low manual production efficiency, reduces dilution costs, improves dilution efficiency, and can avoid contamination on hand of test personnel, meeting the requirements of high-intensity operations.

The embodiment of the present disclosure further provides a filter pressing method, including:
the liquid transfer module 21 drives a liquid transfer component 22 filled with reaction liquid to move above a filter pressing bottle group, and the controller controls the liquid transfer component 22 to inject the reaction liquid into a target filter pressing bottle; the target filter pressing bottle is the first filter pressing bottle or the second filter pressing bottle;
the filter pressing bottle group transfer module 11 drives the filter pressing bottle group carrier 13 to move to the filter pressing position 12, and the filter pressing bottle group carrier 13 is loaded with the second filter pressing bottle; and
the filter pressing transfer module 31 drives a first clamping device 32 to clamp the first filter pressing bottle 14 and move to the filter pressing position 12, cooperate with the second filter pressing bottle 15 that moves to the filter pressing position 12, and apply pressure to the second filter pressing bottle 15 through the first filter pressing bottle 14 to filter press the reaction liquid in the target filter pressing bottle through the filter pressing membrane.

In one embodiment, the filter pressing method includes the following steps:
S1: the liquid transfer module 21 drives a liquid transfer component 22 filled with reaction liquid to move above a filter pressing bottle group, and the controller controls the liquid transfer component 22 to inject the reaction liquid into a target filter pressing bottle; the target filter pressing bottle is the first filter pressing bottle or the second filter pressing bottle;
S2: the filter pressing bottle group transfer module 11 drives the filter pressing bottle group carrier 13 to move to the filter pressing position 12, and the filter pressing bottle group carrier 13 is loaded with the second filter pressing bottle;
S3: the filter pressing transfer module 31 drives a clamping device 32 to clamp the first filter pressing bottle 14 and move to the filter pressing position 12, cooperate with the second filter pressing bottle 15 that moves to the filter pressing position 12, and apply pressure to the second filter pressing bottle 15 through the first filter pressing bottle 14 to filter-press the reaction liquid in the target filter pressing bottle through the filter pressing membrane.

In another embodiment, the filter pressing method includes the following steps:
S1: the filter pressing bottle group transfer module 11 drives the filter pressing bottle group carrier 13 to move to the filter pressing position 12, and the filter pressing bottle group carrier 13 is loaded with the second filter pressing bottle;
S2: the liquid transfer module 21 drives a liquid transfer component 22 filled with reaction liquid to move above a filter pressing bottle group, and the controller controls the liquid transfer component 22 to inject the reaction liquid into a target filter pressing bottle; the target filter pressing bottle is the first filter pressing bottle or the second filter pressing bottle;
S3: the filter pressing transfer module 31 drives a first clamping device 32 to clamp the first filter pressing bottle 14 and move to the filter pressing position 12, cooperate with the second filter pressing bottle 15 that moves to the filter pressing position 12, and apply pressure to the second filter pressing bottle 15 through the first filter pressing bottle 14 to filter-press the reaction liquid in the target filter pressing bottle through the filter pressing membrane.

It should be noted that, the exemplary embodiments mentioned in the present disclosure describe some methods and systems based on a series of operations or devices. However, the present disclosure is not limited to the order of the above operations, and can also choose an appropriate judgment order or perform several operations and steps simultaneously as needed, and this is not limited by the present disclosure.

In one embodiment, the working mode of the filter pressing and analysis operation is as follows: the liquid transfer Y-axis transfer module 212 is driven to move along the X-axis direction through the liquid transfer X-axis transfer module 211; while the liquid transfer Y-axis transfer module 212 moves along the X-axis direction, the liquid transfer Y-axis transfer module 212 also synchronously drives the liquid transfer component 22 to move along the Y-axis direction; the liquid transfer X-axis transfer module 211 and the liquid transfer Y-axis transfer module 212 are linked to drive the liquid transfer component 22 filled with the reaction liquid to move above the filter pressing bottle group; under the control of the controller, the liquid transfer component 22 injects the filled reaction liquid into the target filter pressing bottle, and then the filter pressing bottle group transfer module 11 is driven to the filter pressing bottle group carrier 13 load with the second filter pressing bottle 15 to move to the filter pressing position 12; finally, the filter pressing X-axis transfer module 311 drives the filter pressing Z-axis transfer module 312 to move along the X-axis direction to the filter pressing position 12, and the filter pressing Z-axis transfer module 312 drives the first clamping device 32 to move along the Z-axis direction to clamp the first filter pressing bottle 14, and the first filter pressing bottle and the second filter pressing bottle are coaxial and cooperate with the second filter pressing bottle 15 located at the filter pressing position. The first filter pressing bottle 14 applies pressure to the second filter pressing bottle 15 to filter-press the reaction liquid in the target filter pressing bottle through the filter pressing membrane. After filter pressing is completed, the analysis disk transfer module 16 drives the analysis disk 17 to arrive at the filter pressing bottle group placement position 18, and then the filter pressing Z-axis transfer module 312 drives the first clamping device 32 to move along the Z-axis direction and clamp the filter pressing bottle group on the filter pressing position 12. The filter pressing X-axis transfer module 311 moves to the filter pressing bottle group placement position 18 along the X-axis direction, and places the filter pressing bottle group on the analysis disk 17, then the analysis disk Z-axis transfer module 42 is driven through the analysis disk Y-axis transfer module 41 to move to the filter pressing bottle group placement position 18 along the Y-axis direction, and the analysis disk Z-axis transfer module 42 drives the second clamping device 43 to move along the Z-axis direction to clamp the analysis disk 17 filled with filter pressing bottles, finally the analysis disk 17 clamped with the filter pressing bottle group is driven through the analysis disk Y-axis transfer module 41 to move to the next process, completing analysis and discharge.

The filter pressing system achieves automated connection between multiple processes of filter pressing by provision of a multi-directional transfer module, achieving automated filter pressing; it solves the problem of low manual production efficiency, reduces filter pressing costs, improves filter pressing efficiency, and can avoid hand contamination by test personnel, meeting the requirements of high-intensity operations; multi-module collaboration work with dispersed flow direction shortens the spatial distance between operation steps in the filter pressing process, reduces the transportation and waiting time between operation steps, makes the flow between different operation steps more convenient, significantly improves the working efficiency of the filter pressing process, and can simultaneously meet the requirements of high-throughput experimental processing.

The foregoing are only some embodiments of the present disclosure and are not intended to limit the present disclosure. Any modification, equivalent replacement, improvement, etc. made within the spirit and principles of the present disclosure shall fall into the scope of protection for the present disclosure.

What is claimed is:

1. A filter pressing system, comprising:
a filter pressing bottle group transfer module, provided with a filter pressing position at one end thereof in a Y-axis direction, used to drive a filter pressing bottle group carrier to move in the Y-axis direction, the filter pressing bottle group carrier being used to load a filter pressing bottle group, the filter pressing bottle group comprising a first filter pressing bottle and a second filter pressing bottle, the first filter pressing bottle being connected to the second filter pressing bottle through a filter pressing membrane provided;
a liquid transfer module, used to drive a liquid transfer component filled with reaction liquid to move in a first predetermined direction, the first predetermined direction comprising an X-axis direction and a Y-axis direction, when the liquid transfer component moves above the filter pressing bottle group, a controller controlling the liquid transfer component to inject the reaction liquid into a target filter pressing bottle; the target filter pressing bottle being the first filter pressing bottle or the second filter pressing bottle; and
a filter pressing transfer module, used to drive a first clamping device to move in a second predetermined direction comprising an X-axis direction and a Z-axis direction to clamp the first filter pressing bottle and move to the filter pressing position, cooperate with the second filter pressing bottle that moves to the filter pressing position, and apply pressure to the second filter pressing bottle through the first filter pressing bottle to filter press the reaction liquid in the target filter pressing bottle through the filter pressing membrane.

2. The filter pressing system according to claim 1, the filter pressing system further comprises an analysis disk transfer module, used to drive an analysis disk to move in the Y-axis direction, and the analysis disk transfer module is adjacent to the filter pressing bottle group transfer module parallel to the Y-axis; and
the end of the analysis disk transfer module close to the filter pressing position is a filter pressing bottle group placement position.

3. The filter pressing system according to claim 2, characterized in that, the filter pressing system further comprises an analysis disk discharge component;
the analysis disk discharge component comprises an analysis disk Y-axis transfer module, an analysis disk Z-axis transfer module and a second clamping device, and the second clamping device is provided on the analysis disk Z-axis transfer module;
the analysis disk transfer module is configured to drive the analysis disk to arrive at the filter pressing bottle group placement position;
the filter pressing transfer module drives the filter pressing bottle group to move to the filter pressing bottle group placement position to place the filter pressing bottle group on the analysis disk; and
the analysis disk Y-axis transfer module and the analysis disk Z-axis transfer module are linked to drive the second clamping device to move to the filter pressing bottle group placement position to clamp the analysis disk filled with the filter pressing bottle group to move to the next step.

4. The filter pressing system according to claim 1, a first carrying portion used to carry a first filter pressing membrane is provided at the bottom of the first filter pressing bottle, and the target filter pressing bottle is the second filter pressing bottle in case that the bottom of the second filter pressing bottle is sealed; and
in the case a carrier membrane is embedded at the bottom of the first filter pressing bottle, and a second carrying portion used to carry a second filter pressing membrane is provided away from the bottom of the second filter pressing bottle, the target filter pressing bottle is the first filter pressing bottle.

5. The filter pressing system according to claim 2, there are at least two groups of the filter pressing bottle group transfer modules, and the two groups of filter pressing bottle group transfer modules are adjacent to the analysis disk transfer module parallel to the Y-axis.

6. The filter pressing system according to claim 3, the liquid transfer module comprises a liquid transfer X-axis transfer module and a liquid transfer Y-axis transfer module, and the liquid transfer X-axis transfer module is provided above the filter pressing bottle group transfer module; the liquid transfer Y-axis transfer module is adjacent to the analysis disk Y-axis transfer module parallel to the Y-axis, and the liquid transfer X-axis transfer module and the liquid transfer Y-axis transfer module are linked to drive the liquid transfer component to move above the filter pressing bottle group transfer module.

7. The filter pressing system according to claim 1, the filter pressing transfer module comprises a filter pressing X-axis transfer module and a filter pressing Z-axis transfer module, and the filter pressing X-axis transfer module is provided above the X-axis where the filter pressing position is; the first clamping device is provided on the filter pressing Z-axis transfer module, and the filter pressing X-axis transfer module and the filter pressing Z-axis transfer module are linked to drive the first clamping device to move.

8. The filter pressing system according to claim 1, a liquid receiving component is provided directly below the liquid transfer component, and the liquid receiving component is configured to collect the reaction liquid dripped from the liquid transfer component.

9. The filter pressing system according to claim 8, the liquid receiving component comprises a liquid receiving tank, a driving mechanism, and an installation plate used to connect with the liquid transfer component, and the driving mechanism is configured to drive the liquid receiving tank to move.

10. The filter pressing system according to claim 3, the filter pressing system further comprises a support component, and the support component comprises a support column and a support arm vertically provided with the support column;
the support column is configured to support the liquid transfer module, filter pressing transfer module, analysis disk discharge component and the support arm; and
the liquid transfer module and the analysis disk discharge component are provided on the support arm, and in sliding connection.

11. A filter pressing method, being applied to the filter pressing system according to claim 1, the filter pressing method comprising:
the liquid transfer module drives a liquid transfer component filled with reaction liquid to move above a filter pressing bottle group, and the controller controls the liquid transfer component to inject the reaction liquid into a target filter pressing bottle; the target filter pressing bottle is the first filter pressing bottle or the second filter pressing bottle;
the filter pressing bottle group transfer module drives the filter pressing bottle group carrier to move to the filter pressing position, and the filter pressing bottle group carrier is loaded with the second filter pressing bottle; and
the filter pressing transfer module drives a first clamping device to clamp the first filter pressing bottle and move to the filter pressing position, cooperate with the second filter pressing bottle that moves to the filter pressing position, and apply pressure to the second filter pressing bottle through the first filter pressing bottle to filter press the reaction liquid in the target filter pressing bottle through the filter pressing membrane.

12. A filter pressing system, comprising:
a controller;
a first filter pressing bottle defining a first filter pressing bottle receptacle;
second filter pressing bottle defining a second filter pressing bottle receptacle configured to receive a bottom portion of the first filter pressing bottle during a pressing operation to form a filter pressing bottle assembly comprising the first filter pressing bottle and the second filter pressing bottle and to filter an insoluble substance from an unfiltered reaction fluid disposed in one of the first filter pressing bottle receptacle and the second filter pressing bottle receptacle to produce a filtered reaction fluid that is transferred to the other of the first filter pressing bottle receptacle and the second filter pressing bottle receptacle;
a filter membrane through which the first filter pressing bottle receptacle and the second filter pressing bottle receptacle are in fluid communication in the filter pressing bottle assembly, wherein the filter membrane is configured to filter out the insoluble substance from the unfiltered reaction fluid;
a filter pressing bottle carrier comprising a first filter pressing bottle support receptacle configured to receive and support the first filter pressing bottle and a second filter pressing bottle support receptacle configured to receive and support the second filter pressing bottle;
an analysis disk comprising a filter pressing bottle assembly receptacle configured to receive and support the filter pressing bottle assembly;
a filter pressing transfer module operable by the controller to:
grasp and remove the first filter pressing bottle from the first filter pressing bottle support receptacle,
align and insert the first filter pressing bottle into the second filter pressing bottle to accomplish the pressing operation, and
move and insert the filter pressing bottle assembly into the filter pressing bottle receptacle; and
an analysis disk discharge assembly operable by the controller to grasp the analysis disk and reposition the analysis disk for discharge of the analysis disk.
